(19) United States
(12) Patent Application Publication
(10) Pub. No.: US 2019/0262307 A1
(43) Pub. Date: Aug. 29, 2019

(54) IGURATIMOD AS AN MIF INHIBITOR

(71) Applicant: THE FEINSTEIN INSTITUTE FOR MEDICAL RESEARCH, Manhasset, NY (US)

(72) Inventors: Yousef Al-Abed, Dix Hills, NY (US); Joshua Bloom, Oakland Gardens, NY (US); Mohamed Ahmed, Commack, NY (US)

(73) Assignee: THE FEINSTEIN INSTITUTE FOR MEDICAL RESEARCH, Manhasset, NY (US)

(21) Appl. No.: 16/348,579

(22) PCT Filed: Nov. 8, 2017
(86) PCT No.: PCT/US2017/060498

§ 371 Date: May 9, 2019

Related U.S. Application Data
(60) Provisional application No. 62/419,530, filed on Nov. 9, 2016.

(51) Int. Cl. A61K 31/352; A61P 21/00

(52) U.S. Cl. CPC A61K 31/352; A61P 21/00

(57) ABSTRACT

Methods of treatment of MIF-related diseases and methods of MIF inhibition are provided.

### CROSS-REFERENCE TO RELATED APPLICATIONS

This application claims benefit of U.S. Provisional Application No. 62/419,530, filed Nov. 9, 2016, the contents of which are hereby incorporated by reference.

### BACKGROUND OF THE INVENTION

Throughout this application various patents and other publications are referred to by number in parenthesis. Full citations for the references may be found at the end of the specification. The disclosures of these references and all patents, patent application publications and books referred to herein are hereby incorporated by reference in their entirety into the subject application to more fully describe the art to which the subject invention pertains.

Macrophage migration inhibitory factor (MIF) is a pleiotropic cytokine that has been implicated in a broad range of inflammatory and oncologic disease conditions. MIF is unique among cytokines in terms of its release profile and inflammatory role, notably as an endogenous counter-regulator of the anti-inflammatory effects of glucocorticoids. In addition, it possesses a catalytic tautomerase activity amenable to the design of highly affine small molecule inhibitors. Although several classes of these compounds have been identified, few have been well characterized biologically, and notably no studies have been undertaken examining the off-target effects of these molecules. Novel MIF inhibitors are of great interest for clinical application in MIF-relevant diseases.

The present invention addresses the need for novel MIF inhibitors for treatments of MIF-mediated disease states.

### SUMMARY OF THE INVENTION

A method is provided of inhibiting macrophage migration inhibitory factor (MIF) in a subject comprising administering to the subject an amount of the following compound, or a pharmaceutically acceptable salt thereof, effective to inhibit MIF:

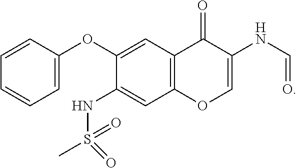 [SMILES: CS(=O)(=O)Nc1cc2occ(NC=O)c(=O)c2cc1Oc1ccccc1]

Also provided is a method of inhibiting macrophage migration inhibitory factor (MIF) comprising contacting the MIF with an amount of the following compound, or a pharmaceutically acceptable salt thereof, effective to inhibit MIF:

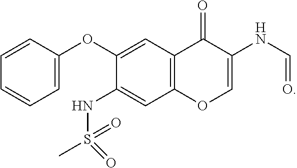 [SMILES: CS(=O)(=O)Nc1cc2occ(NC=O)c(=O)c2cc1Oc1ccccc1]

Also provided is a method of treating a disease mediated by macrophage migration inhibitory factor (MIF) in a subject, comprising administering to the subject an amount of the following compound, or a pharmaceutically acceptable salt thereof, effective to treat the disease:

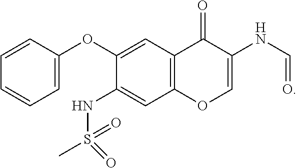 [SMILES: CS(=O)(=O)Nc1cc2occ(NC=O)c(=O)c2cc1Oc1ccccc1]

Also provided is a method of reducing the dose of a steroid administered to a subject required to achieve a therapeutic effect in a disease treatable by steroid therapy, comprising administering to the subject having the disease an amount of a compound or a pharmaceutically acceptable salt of such compound effective to reduce the dose of steroid needed to achieve the therapeutic effect in the disease, wherein the compound has the following structure:

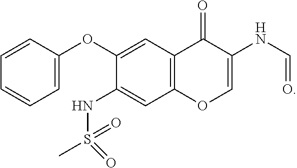 [SMILES: CS(=O)(=O)Nc1cc2occ(NC=O)c(=O)c2cc1Oc1ccccc1]

Also provided is a method of increasing the likelihood of success of a liver transplant or kidney transplant in a subject comprising administering to the subject an amount of the following compound, or a pharmaceutically acceptable salt thereof, effective to increase the likelihood of success of a liver transplant or kidney transplant in a subject:

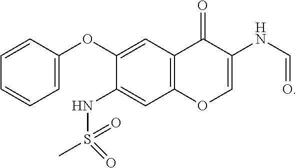 [SMILES: CS(=O)(=O)Nc1cc2occ(NC=O)c(=O)c2cc1Oc1ccccc1]

A method is provided for treating congenital diaphragmatic hernia in a subject, comprising administering to the subject an amount of the following compound, or a pharmaceutically acceptable salt thereof, effective to treat the disease:

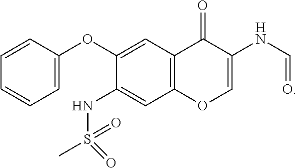 [SMILES: CS(=O)(=O)Nc1cc2occ(NC=O)c(=O)c2cc1Oc1ccccc1]

Also provided is a composition comprising a pharmaceutically acceptable carrier, an amount of acetaminophen, and an amount of a compound having the structure set forth below, or a pharmaceutically acceptable salt of such compound:

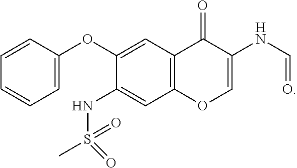 [SMILES: CS(=O)(=O)Nc1cc2occ(NC=O)c(=O)c2cc1Oc1ccccc1]

Also provided is a solid composition comprising (i) from 300 to 1500 mg of acetaminophen, and (ii) an amount of a compound having the structure set forth below, or a pharmaceutically acceptable salt of such compound, effective to reduce acetaminophen hepatotoxicity in a human subject, wherein the compound has the following structure:

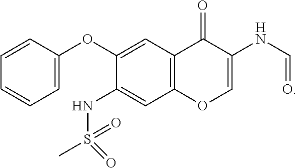 [SMILES: CS(=O)(=O)Nc1cc2occ(NC=O)c(=O)c2cc1Oc1ccccc1]

Also provided is a liquid composition comprising (i) from 20 mg per ml to 150 mg per ml of acetaminophen, and (ii) an amount of a compound having the structure set forth below, or a pharmaceutically acceptable salt of such compound, effective to reduce acetaminophen hepatotoxicity in a human subject, wherein the compound has the following structure:

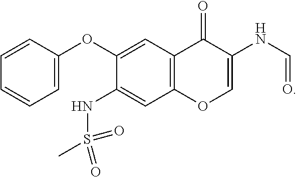 [SMILES: CS(=O)(=O)Nc1cc2occ(NC=O)c(=O)c2cc1Oc1ccccc1]

Also provided is a method of decreasing the likelihood of hepatotoxicity in a subject resulting from an overdose of acetaminophen, comprising administering to the subject one or more doses of the acetaminophen composition(s) described herein amounting to more than 3 g of acetaminophen in 24 hours, wherein the amount of the compound or pharmaceutically acceptable salt of such compound is effective to decrease the likelihood of hepatotoxicity in a subject resulting from an overdose of acetaminophen.

Also provided is a composition comprising a pharmaceutically acceptable carrier, an amount of acetaminophen, and an amount of an MIF inhibitor compound effective to ameliorate hepatotoxic effects of the acetaminophen.

Also provided is a method of treating hepatotoxicity in a subject who is suffering from an overdose of amount of acetaminophen, comprising administering to the subject an amount of an MIF inhibitor compound effective to ameliorate hepatotoxic effects of acetaminophen overdose.

Additional objects of the invention will be apparent from the description which follows.

### BRIEF DESCRIPTION OF THE DRAWINGS

FIG. 1A-1D: MIF Inhibitors have distinct anti-inflammatory profiles, which are MIF-independent. (1A) and (1B): human peripheral blood monocytes purified by negative selection were pre-treated with the indicated dose of MIF inhibitor for 1 hour prior to 24-hour stimulation with 1 ng/mL LPS from E. coli R515, with results converted to percentage of maximum cytokine release and shown as an average of two independent experiments. MIF inhibitors showed at least three patterns of anti-TNFα activity (1A) that are mostly consistent upon examination of chemokines MCP-1 and IL-8 (1B). (1C) and (1D): murine peripheral blood leukocytes from 3-4 animals per experiment were plated directly from Ficoll-isolated buffy coats, pre-treated with inhibitor for 1 hour where applicable, and stimulated with 1 ng/mL LPS from E. coli R515 for 5 hours. Results are shown mean/SEM. (1C), MIF KO animals show no differences in levels of induced TNFα (unpaired one-tailed t-test, p=0.2358) or IL-6 (t-test, p=0.3768) in response to LPS. (1D), K-680 demonstrates anti-TNFα activity in both wild-type and MIF−/− leukocytes. A two-way ANOVA was performed with a Bonferroni correction; *** p=0.0008, * p=0.0486.

FIG. 2A-2B: T-614 exhibits MIF-specific inhibition in vitro. (2A) Human Raji B cells were synchronized for 24 hours in 0.1-0.5% FBS, pre-treated with the indicated doses of T-614 for 20 minutes, and then stimulated with 1 ng/mL rMIF or control for 24 hours with BrdU added at 20 hours. Results are expressed as absorbance values from a BrdU incorporation kit (Cell Signaling Technologies) and are representative of two independent experiments using quadruplicate samples. Results are shown mean+SEM, and a one-way ANOVA was performed with indicated comparisons selected for a Bonferroni correction: ns, p=0.3346; ***, p=0.0006; **, p=0.0024. B, adherence-purified human peripheral blood monocytes were pre-treated with indicated doses of T-614 prior to stimulation with 100 ng/mL rMIF. (2B) Adherence-purified human peripheral blood monocytes were pre-treated with indicated doses of T-614 for 20 minutes, and then stimulated with 100 ng/mL rMIF or control for 24 hours before analysis with IL-8 ELISA. Results shown are representative of three independent experiments using quadruplicate samples. Results are shown mean+SEM, and a one-way ANOVA was performed with indicated comparisons selected for a Bonferroni correction: ns, p>0.9999; ***, p<0.0001; *, p=0.0307.

FIG. 3A-3C: T-614 exhibits MIF-specific inhibition in vivo. (3A) Male BALB/c mice (n=10/group) were treated with 5 mg/kg LPS from E. coli O111:B4 to induce lethal endotoxemia, and monitored for survival over 2 weeks. Survival data were analyzed using a Log-rank test, p=0.031. (3B) and (3C), male C57/BL6 ((3B), n=5/group) and matched MIF^{-/-} mice (3C), n=6/group) were administered a non-lethal dose of LPS and sacrificed at 90 minutes for tissue collection. Data are shown mean±SEM with individual subjects indicated, and were analyzed using unpaired one-tailed t-tests: (3B), * p=0.0078; (3C), ns p=0.3965.

FIG. 4A-4D: T-614 has additive anti-inflammatory effects when used with glucocorticoids in vitro. All cells were pre-treated 20 minutes with T-614 or vehicle (PBS pH 7.8), 20 minutes with dexamethasone or vehicle (0.01% DMSO), and then stimulated with the indicated dose of LPS from E. coli O111:B4. Cell-free supernatants were collected after the indicated time period and subjected to a TNFα ELISA. (4A) RAW 264.7 cells were treated using 100 μM T-614, 50 nM dexamethasone, 0.1 ng/mL LPS, and a 4-hour stimulation with LPS. (4B) THP-1 cells were treated using 100 μM T-614, 50 nM dexamethasone, 5 ng/mL LPS, and a 16-hour stimulation with LPS. (4C) adherence-purified human peripheral blood monocytes were treated using 100 μM T-614, 50 nM dexamethasone, 1 ng/mL LPS, and a 16-hour stimulation with LPS. (4D) M-CSF polarized macrophages were treated using 200 μM T-614, 50 nM dexamethasone, 0.5 ng/mL LPS, and a 16-hour stimulation with LPS. All results are shown mean+SEM, and are representative of three independent experiments using samples in quadruplicate. Data were analyzed using a one-way ANOVA with a Bonferroni correction: A, *(1) p=0.0130; *(2) p=0.0417; ** p=0.0088. B,*** p<0.0001. C, *** p<0.0005. D, *** p<0.0001; * p=0.0107.

FIG. 5: A significant increase of eNOS phosphorylation was induced by MIF inhibitors ISO-1, ISO-92, and the proposed MIF inhibitor T614/iguratimod in comparison to Nitrofen group among neonates pups with CDH (P<0.05).

FIG. 6: A significant increase of VGEF was seen among neonates pups with CDH and treated by MIF inhibitors ISO-1, ISO-92, and the proposed MIF inhibitor T614/iguratimod in comparison to Nitrofen group with CDH (P<0.05).

FIG. 7: A significant reduction of Arginase I was seen among treated pups with CDH with MIF inhibitors (including T614/iguratimod) compared to Nitrofen group (P<0.05).

FIG. 8: Arginase II was reduced in all treated groups (ISO-1, ISO-92, and the proposed MIF inhibitor T614/iguratimod) but was significantly lower among ISO-92 and ISO-1 only (P<0.05) (FIG. 8).

FIG. 9: Liver H₂O₂ content in APAP-treated rats treated with T614/iguratimod and control determined using a hydrogen peroxide assay.

FIG. 10: Survival rates of APAP-treated rats treated with T614/iguratimod and control.

### DETAILED DESCRIPTION OF THE INVENTION

A method is provided for inhibiting macrophage migration inhibitory factor (MIF) in a subject comprising administering to the subject an amount of the following compound, or a pharmaceutically acceptable salt thereof, effective to inhibit MIF:

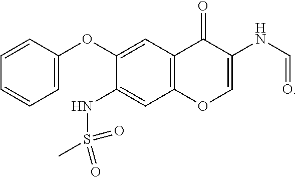 [SMILES: CS(=O)(=O)Nc1cc2occ(NC=O)c(=O)c2cc1Oc1ccccc1]

In an embodiment, the subject has congenital diaphragmatic hernia. In an embodiment, the subject does not have an autoimmune disease. In an embodiment, the subject does not have a nonsterile inflammatory disease. In an embodiment, the subject does not have a sterile inflammatory disease.

A method is provided for inhibiting macrophage migration inhibitory factor (MIF) comprising contacting the MIF with an amount of the following compound, or a pharmaceutically acceptable salt thereof, effective to inhibit MIF:

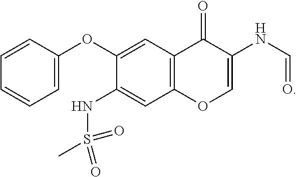 [SMILES: CS(=O)(=O)Nc1cc2occ(NC=O)c(=O)c2cc1Oc1ccccc1]

A method is provided for treating congenital diaphragmatic hernia in a subject, comprising administering to the subject an amount of the following compound, or a pharmaceutically acceptable salt thereof, effective to treat the disease:

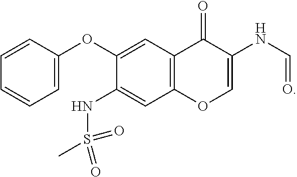 [SMILES: CS(=O)(=O)Nc1cc2occ(NC=O)c(=O)c2cc1Oc1ccccc1]

Also provided is a method for treating a disease mediated by macrophage migration inhibitory factor (MIF) in a subject, comprising administering to the subject an amount of the following compound, or a pharmaceutically acceptable salt thereof, effective to treat the disease:

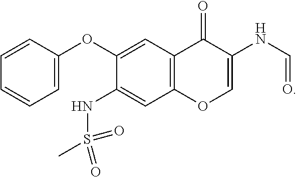 [SMILES: CS(=O)(=O)Nc1cc2occ(NC=O)c(=O)c2cc1Oc1ccccc1]

In an embodiment, the disease is not an autoimmune disease and/or is not an inflammatory disease.

In an embodiment, the disease is congenital diaphragmatic hernia.

Also provided is a method of reducing the dose of a steroid administered to a subject required to achieve a predetermined therapeutic effect in a disease treatable by steroid therapy, comprising administering to the subject having the disease an amount of a compound or a pharmaceutically acceptable salt of such compound effective to reduce the dose of steroid needed to achieve the therapeutic effect in the disease, wherein the compound has the following structure:

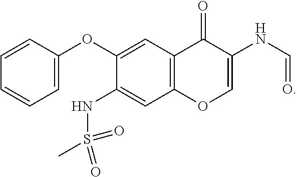 [SMILES: CS(=O)(=O)Nc1cc2occ(NC=O)c(=O)c2cc1Oc1ccccc1]

Also provided is a method of increasing the likelihood of success of an organ transplant in a subject comprising administering to the subject an amount of the following compound, or a pharmaceutically acceptable salt thereof, effective to increase the likelihood of success of an organ transplant in a subject:

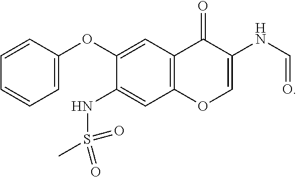 [SMILES: CS(=O)(=O)Nc1cc2occ(NC=O)c(=O)c2cc1Oc1ccccc1]

In an embodiment, the organ is a liver or a kidney. In an embodiment, the organ is a liver. In an embodiment, the organ is a kidney.

In an embodiment of the methods described herein, the compound is administered. In an embodiment of the methods described herein, the pharmaceutically acceptable salt of the compound is administered.

In an embodiment of the methods described herein, the subject is a human.

Also provided is a composition comprising a pharmaceutically acceptable carrier, an amount of acetaminophen, and an amount of a compound having the structure set forth below, or a pharmaceutically acceptable salt of such compound:

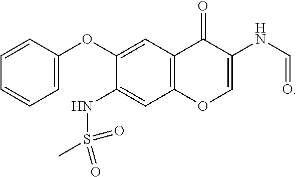 [SMILES: CS(=O)(=O)Nc1cc2occ(NC=O)c(=O)c2cc1Oc1ccccc1]

Also provided is a solid composition comprising (i) from 300 to 1500 mg of acetaminophen, and (ii) an amount of a compound having the structure set forth below, or a pharmaceutically acceptable salt of such compound, effective to reduce acetaminophen hepatotoxicity in a human subject, wherein the compound has the following structure:

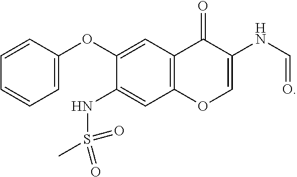 [SMILES: CS(=O)(=O)Nc1cc2occ(NC=O)c(=O)c2cc1Oc1ccccc1]

Also provided is a liquid composition comprising (i) from 20 mg per ml to 150 mg per ml of acetaminophen, and (ii) an amount of a compound having the structure set forth below, or a pharmaceutically acceptable salt of such compound, effective to reduce acetaminophen hepatotoxicity in a human subject, wherein the compound has the following structure:

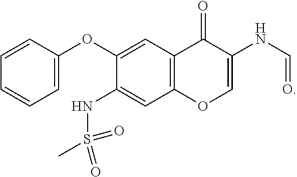 [SMILES: CS(=O)(=O)Nc1cc2occ(NC=O)c(=O)c2cc1Oc1ccccc1]

In an embodiment of the compositions, the composition further comprises a pharmaceutically acceptable carrier.

Also provided is a method of decreasing the likelihood of hepatotoxicity in a subject resulting from an overdose of acetaminophen, comprising administering to the subject one or more doses of the acetaminophen composition(s) described herein amounting to more than 3 g of acetaminophen in 24 hours, wherein the amount of the compound or pharmaceutically acceptable salt of such compound is effective to decrease the likelihood of hepatotoxicity in a subject resulting from an overdose of acetaminophen.

In an embodiment of the methods an compositions described herein, the acetaminophen is N-(4-hydroxyphenyl)ethanamide or N-(4-hydroxyphenyl)acetamide.

Also provided is a composition comprising a pharmaceutically acceptable carrier, an amount of acetaminophen, and an amount of an MIF inhibitor compound effective to ameliorate hepatotoxic effects of the acetaminophen. In an embodiment, the MIF inhibitor is ISO-1. In an embodiment, the MIF inhibitor is ISO-92. In an embodiment, the MIF inhibitor is T614.

Also provided is a method of treating hepatotoxicity in a subject who is suffering from an overdose of amount of acetaminophen, comprising administering to the subject an amount of an MIF inhibitor compound effective to ameliorate hepatotoxic effects of acetaminophen overdose. In an embodiment the compound has the structure set forth below, or is a pharmaceutically acceptable salt of such compound:

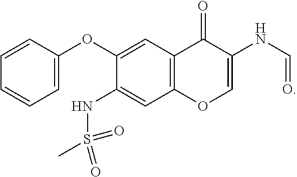 [SMILES: CS(=O)(=O)Nc1cc2occ(NC=O)c(=O)c2cc1Oc1ccccc1]

In an embodiment of the methods and compositions described herein, the composition comprises the compound. In an embodiment of the methods and compositions described herein, the composition comprises the pharmaceutically acceptable salt of the compound.

In an embodiment of the methods described herein, the subject is a human.

In general, the amount of an agent “effective” (e.g., a therapeutic agent, composition, and/or formulation such as the agent or a composition comprising the agent) is an amount effective to achieve a stated effect or to elicit the desired biological response. As will be appreciated by those of ordinary skill in this art, an effective amount of a substance may vary depending on such factors as the desired biological endpoint, the substance to be delivered, the pharmacokinetics of the compound, the target cell or tissue, the disease being treated, the mode of administration, and the patient, etc. For example, the effective amount of a composition and/or formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. Those of ordinary skill in the art will appreciate that, commonly, an effective amount will be administered over a series of individual doses. In some embodiments, the term “effective amount” when used in a pharmaceutical context (e.g., pharmaceutically effective amount) means that an agent is present in an amount sufficient to achieve a desired therapeutic effect.

Routes of administration, unless otherwise specified, encompassed by the methods of the invention include, but are not limited to, each of the following individual routes, and any subset thereof, auricular, buccal, conjunctival, cutaneous, subcutaneous, endocervical, endosinusial, endotracheal, enteral, epidural, via hemodialysis, interstitial, intrabdominal, intraamniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronary, intradermal, intradiscal, intraductal, intraepidermal, intraesophagus, intragastric, intravaginal, intragingival, intraileal, intraluminal, intralesional, intralymphatic, intramedullary, intrameningeal, intramuscular, intraocular, intraovarian, intraepicardial, intraperitoneal, intraplacental, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intraventricular, intravesical, intravitreal, laryngeal, nasal, nasogastric, ophthalmic, oral, oropharyngeal, parenteral, percutaneous, periarticular, peridural, rectal, inhalationally, retrobulbar, subarachnoid, subconjuctival, sublingual, submucosal, topically, transdermal, transmucosal, transplacental, transtracheal, ureteral, uretheral, and vaginal administration.

In embodiments, the subject does not have arthritis. In embodiments, the subject has not been diagnosed with arthritis. In embodiments, the subject has not been treated for arthritis.

In an embodiment of the methods, the subject is human.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Results

### Introduction

In this study, in vitro assays were used to characterize representative molecules from several classes of MIF inhibitors. It was determined that MIF inhibitors exhibit distinct profiles of anti-inflammatory activity, and that these activities can be MIF-independent. It was further investigated if a molecule with low off-target anti-inflammatory activity, compound T-614 (also known as the anti-rheumatic drug, iguratimod), was a selective MIF inhibitor. It was found that in addition to inhibiting MIF-specific activities in vitro and in vivo, iguratimod also has additive effects with glucocorticoids in inflammatory contexts.

### Results

Compounds from multiple classes of MIF small-molecule inhibitors (detailed in Table 1) as well as previously unknown MIF inhibitors were selected for testing as broad anti-inflammatory compounds in an LPS-treated monocyte system. It was discovered that, even within the small cohort, compounds segregated into at least three groups with distinct anti-inflammatory profiles. However, compounds with a high anti-inflammatory profile maintained these effects even in MIF^{-/-} cells; this led to a conclusion that the anti-inflammatory effects tested in the study are MIF-independent effects, and that MIF inhibitors with a low anti-inflammatory profile are potentially MIF-selective. Using MIF-dependent in vitro and in vivo studies, it was determined that the chromene-derived compound T-614—better known as the anti-rheumatic drug, iguratimod—inhibits MIF and attenuates inflammatory disease in an MIF-dependent fashion, and that its anti-inflammatory effects are additive with glucocorticoids. The data suggests that iguratimod can exert clinical anti-inflammatory activities via MIF inhibition. This drug can be used in a steroid-sparing therapy.

TABLE 1
Representative MIF inhibitory compounds selected for characterization.
IC₅₀ values are based on MIF dopachrome tautomerase activity
as detailed in Materials and Methods.

| | | | IC₅₀, μM | |
|---|---|---|---|---|
| Compound | Category | Structure | (±SD) | Reference |
| ISO-1 | Isoxazole | 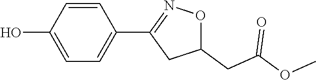 [SMILES: COC(=O)CC1CC(c2ccc(O)cc2)=NO1] | 18.20 ± 2.90 | Lubetsky 2002 |
| ISO-66 | Isoxazole | 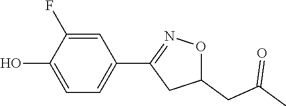 [SMILES: CC(=O)CC1CC(c2ccc(O)c(F)c2)=NO1] | 1.47 ± 0.44 | Ioannou 2014 |
| ISO-92 | Isoxazole | 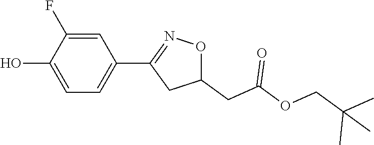 [SMILES: CC(C)(C)COC(=O)CC1CC(c2ccc(O)c(F)c2)=NO1] | 1.07 ± 0.01 | Novel |
| K-679 | Coumarin | 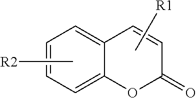 | 0.22 ± 0.04 | Novel |
| K-680 | Coumarin | 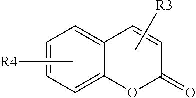 | 1.12 ± 0.04 | Novel |
| T-614 | Chromene | 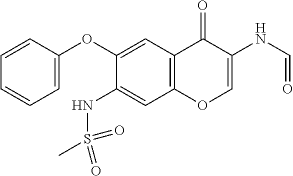 [SMILES: CS(=O)(=O)Nc1cc2occ(NC=O)c(=O)c2cc1Oc1ccccc1] | 6.81 ± 0.56 | Novel |
| K-664.1 | Pyrimidazole | 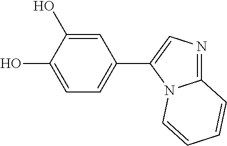 [SMILES: Oc1ccc(-c2cnc3ccccn23)cc1O] | 0.16 ± 0.06 | Novel |
| OXIM-11 | Carbonyl oxime | 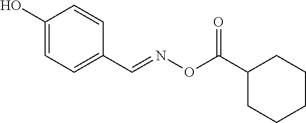 [SMILES: O=C(O/N=C/c1ccc(O)cc1)C1CCCCC1] | 1.57 ± 0.15 | Crichlow 2007 |
| d-T4 | Hormone isomer | 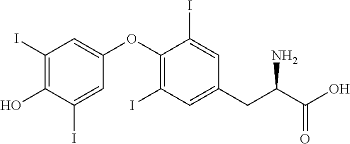 [SMILES: N[C@H](Cc1cc(I)c(Oc2cc(I)c(O)c(I)c2)c(I)c1)C(=O)O] | 11.30 ± 0.29 | Al-Abed 2011 |

### Materials and Methods

Reagents: All reagents were purchased from Sigma-Aldrich or Fisher Scientific unless otherwise indicated. Compound T-614 (iguratimod) was purchased from Ontario Chemical (Guelph, Ontario) and solubilized in alkaline solution (pH 7.8) for in vitro and in vivo studies. Recombinant human MIF protein for catalytic characterization and in vitro use was expressed in E. coli BLD1(DE3) cells and purified as described previously (25); in vitro experiments were confirmed (where applicable) with bioactive recombinant human MIF purchased from Shenanodah Biotechnologies (Warwick, Pa.). Prior to in vitro use endotoxin content was confirmed to be less than 0.05 EU/μg protein by a colorimetric endpoint LAL assay (Lonza; Allendale, N.J.). Cytokine ELISAs were purchased as DuoSet kits from R&D Systems (unless otherwise indicated) and used according to the manufacturer's instructions (Minneapolis, Minn.).

MIF Dopachrome Tautomerase Activity: The enzymatic activity of MIF on freshly prepared L-dopachrome methyl ester was assayed as described previously (33). Sterile recombinant MIF was maintained in TBS pH 7.4 at concentrations ranging from 0.5-1 mg/mL for up to six months without significant loss of enzymatic activity. Inhibitory compounds were solubilized in DMSO, added to a cuvette containing 1 μg/mL rMIF in PBS and mixed thoroughly; dopachrome substrate was then added and absorbance at 475 nm was monitored for 20 seconds to measure activity.

Cell Culture: Human Raji B and THP-1 cells were maintained in suspension culture in RPMI 1640 supplemented with 10% heat-inactivated fetal bovine serum, 100 units/mL penicillin-streptomycin, 2 mM L-glutamine, and 55 μM 2-mercaptoethanol (THP-1 cells only) (Life Technologies; Carlsbad, Calif.). Cells were passaged by dilution three times weekly and total media replacement every three weeks. Raji B cells were used for two months post-thaw, and THP-1 cells were used only during weeks 2 and 3 post-thaw. RAW 264.7 macrophages were maintained in adherent culture in DMEM/4.5 g/dL glucose supplemented with 10% heat-inactivated fetal bovine serum, 100 units/mL penicillin-streptomycin, and 2 mM L-glutamine; cells were passaged by scraping and used only until passage 20. All media variants contained 100 units/mL penicillin-streptomycin and 2 mM L-glutamine. All cells were cultured in a humidified incubator at 37° C./5% CO₂. Unless otherwise indicated, all cells were purchased from the American Tissue Culture Collection and stored as passage 5 aliquots in liquid nitrogen (ATCC; Manassas, Va.).

Preparation of Peripheral Blood Cells: Human peripheral blood was collected in sodium heparin (IRB#12-200A) or obtained as Leukopaks from New York Blood Center (New York, N.Y.). Mononuclear cells were isolated by density gradient centrifugation in Ficoll-Paque Plus (GE Healthcare; Pittsburgh, Pa.). Monocytes were either purified by two-hour adherence to Primaria culture plates (Corning Life Sciences; Corning, N.Y.) or enriched by negative magnetic selection using Monocyte Isolation Kit II (Miltenyi Biotech; Auburn, Calif.). All monocyte preps were cultured in RPMI supplemented with 10% human AB serum, 100 units/mL penicillin-streptomycin, and 2 mM L-glutamine and used within 24 hours. Macrophages were differentiated from adherence-purified monocytes by incubation with 10 ng/mL human M-CSF (Sigma) for seven days, with media replenishment performed on days 3 and 5.

Mouse peripheral blood was collected by cardiac puncture with a heparinized needle from animals euthanized by CO₂ asphyxiation. Blood from 4-5 animals was pooled and subjected to density gradient centrifugation in Ficoll-Paque Plus to isolate leukocytes. This laboratory has observed that murine peripheral blood cells are difficult to purify and experience rapid losses in viability over 24 hours; therefore mixed peripheral blood leukocyte populations were plated immediately after isolation and used immediately.

Cytokine Production Assays: For LPS-induced cytokine release, negative selection-enriched monocytes (human) or mixed peripheral blood leukocytes (murine) were plated at a density of 2×10⁵ cells/mL in 96-well plates. Cells were pre-treated with MIF inhibitors in 0.1% DMSO (final) for 1 hour before stimulation with LPS from E. coli R515 (Axxora; Farmingdale, N.Y.): 24 hours for human cells, 5 hours for murine cells. Cell-free supernatants were collected and stored at −80° C. for cytokine determinations by ELISA; 1:2 dilutions were used for TNFα and 1:3 dilutions for IL-8 and MCP-1. Cytotoxicity was assessed using a Cytotox96 Non-Radioactive Cytotoxicity Kit (Promega; Madison, Wis.); no significant cytotoxicity was observed for the compounds used unless otherwise indicated.

Although the production of cytokines by MIF stimulation alone is controversial, the production of IL-8 by stimulation of peripheral blood monocytes has been confirmed in multiple contexts (35, 37). Human peripheral blood monocytes purified by adherence were plated at a density of 2×10⁵ cells/well in a 96-well plate. Cells were pre-treated with T-614 or vehicle for 30 minutes prior to stimulation with MIF for 24 hours. Cell-free supernatants were recovered and subjected to IL-8 MAX Standard ELISA using a 1:100 dilution (Biolegend, San Diego, Calif.). Cytotoxicity was assessed by a Neutral Red assay as previously described, using a 1-hour incorporation period (38); no significant cytotoxicity effects were observed unless otherwise indicated.

MIF-induced Proliferation: For this bioassay we adapted the method of Leng et al. (5) with some modifications. Briefly, human Raji B cells were plated at a density of 0.5×10⁴ cells/well in a 96-well plate and synchronized by incubation for 24 hours in RPMI 1640 supplemented with 0.1-0.5% FBS. Synchronized cells were pre-treated with T-614 or vehicle for 30 minutes prior to stimulation with MIF for 24 hours. At 20 hours BrDU was added to cells and quantified using a BrDU Cell Proliferation Assay Kit (Cell Signaling Technology; Danvers, Mass.).

Glucocorticoid Synergy in vitro: The methods of Roger et al. and Kerschbaumer et al. were adapted for these experiments (17, 21). RAW 264.7 cells were plated at a density of 1×10⁵ cells/well in a 96-well plate and incubated overnight (16 hours) before pretreatment with T-614 or vehicle for 20 minutes, treatment with dexamethasone (Alfa Aesar; Ward Hill, Mass.) solubilized in 0.01% DMSO (final) for 20 minutes, and finally stimulation with LPS from E. coli O111:B4 (Sigma) for 4 hours. Cell-free supernatants were collected and analyzed immediately with a murine TNFα ELISA at a 1:10 dilution. THP-1 cells were plated at a density of 2×10⁵ cells/well in a 96-well plate and used the same day with the same treatment conditions as RAW 264.7 cells, except that THP-1 cells were incubated with LPS for 16 hours prior to isolation of cell-free supernatants. Supernatants were analyzed using a human TNFα ELISA at a 1:10 dilution. Human peripheral blood mononuclear cells isolated by adherence (described above) were plated at a density of 2×10⁵ cells/well in a 96-well plate and used the same day with the same treatment conditions as THP-1 cells. Supernatants were analyzed using a human TNFα ELISA at a 1:20 dilution. Human macrophages were differentiated from adherence-purified monocytes by 1-week culture with 10 ng/mL M-CSF on cells plated at a density of 2×10⁵ cells/well and used when an astrocytic morphology was clearly observed in >50% of the cells. Cells were treated under the same conditions as THP-1 cells and supernatants were analyzed using a human TNFα ELISA at a 1:10 dilution.

For all above experiments, cytotoxicity was assessed using an MTT assay (1-2 hour incorporation); no significant cytotoxicity effects were observed under any treatment conditions unless otherwise noted.

Animal Experiments: The Institutional Animal Care and Use Committee of the Feinstein Institute for Medical Research reviewed and approved all animal protocols prior to initiation of experiments. Male BALB/c mice were purchased from the Jackson Laboratory (Bar Harbor, Me.) and used for endotoxemia experiments at ages 8-10 weeks. MIF KO animals were maintained on a C57/BL6 NCr background from Charles River Laboratories (Stone Ridge, N.Y.); these animals were used alongside matched wild-type animals for endotoxemia experiments at ages 8-12 weeks.

Endotoxemia: Endotoxemia was induced by intraperitoneal injection of LPS from E. coli O111:B4 (Sigma). In BALB/c animals, 5 mg/kg LPS was used as a lethal dose for survival experiments: animals were treated with T-614 (20 mg/kg i.p.) 0.5 hours prior to LPS, 6 hours after LPS, and then once daily for three days, and monitored for survival over two weeks. In C57/BL6 animals, 20 mg/kg LPS was used as non-lethal dose for plasma cytokine experiments: animals were pre-treated with T-614 (20 mg/kg i.p.) twice, one dose each at 2 hours and 0.5 hours prior to LPS administration, and euthanized at 90 minutes post-LPS by CO₂ asphyxiation with cervical dislocation. Blood was collected by cardiac puncture and allowed to clot 20 minutes at room temperature and 20 minutes at 4° C.; sera were isolated by centrifugation at 300×g for 10 minutes and stored at −20° C. for further analysis by TNFα ELISA (1:3 dilution).

### Results

### Example 1

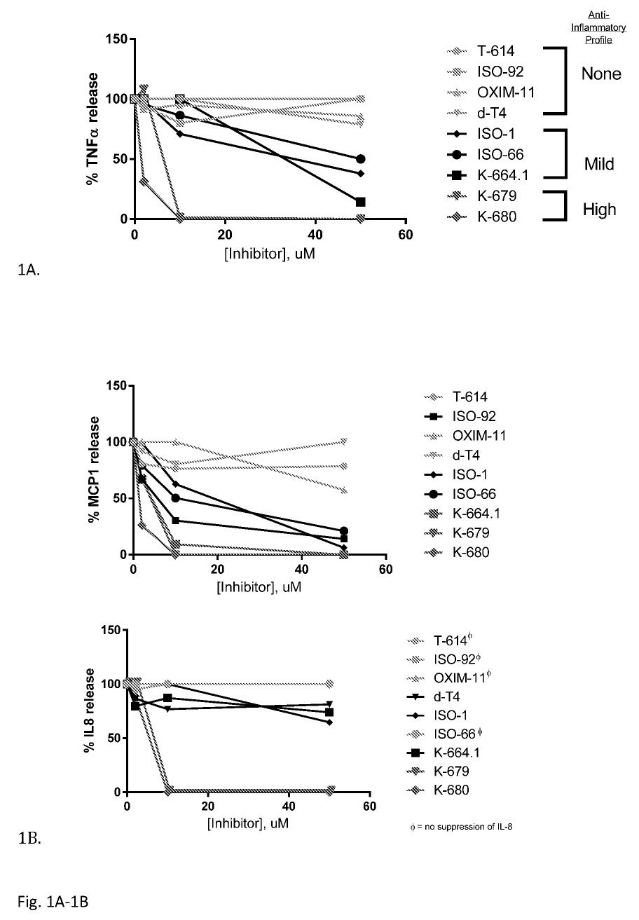

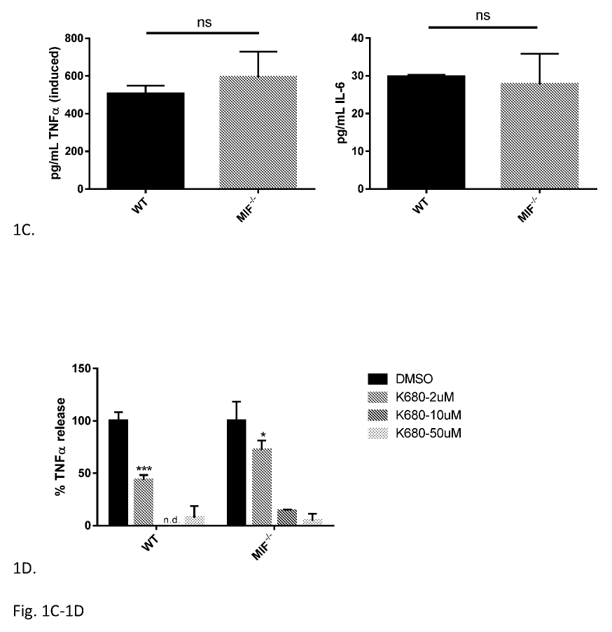

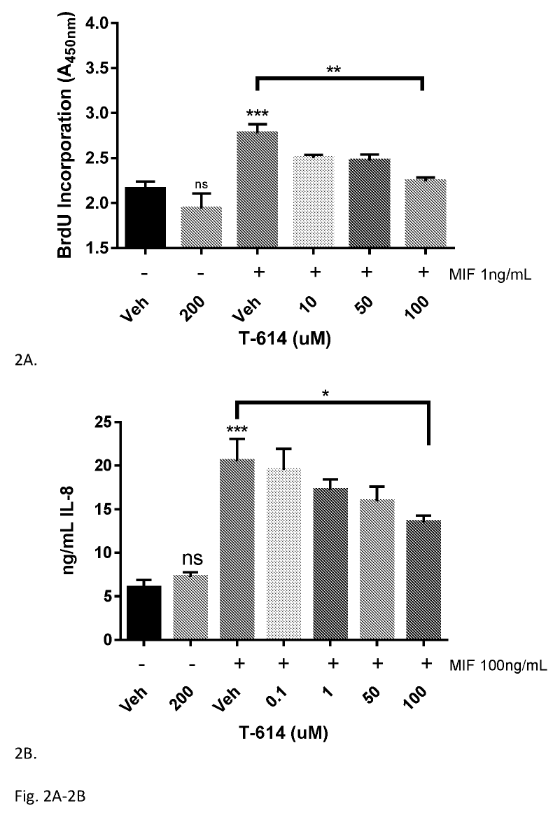

Cytokine Release by Monocytes: an MIF-Independent Effect—Small molecules of various classes with significant IC₅₀ in the MIF tautomerase assay were selected for biological characterization in the context of LPS-treated human monocytes (Table 1). None of these compounds exhibited significant toxicity up to 50 μM in this context (data not shown). It was found that although these molecules all have a similar profile of inhibition of MIF enzymatic activity, they exhibit diverse profiles of anti-inflammatory activity in this bioassay. The clearest distinctions were observable in TNFα release: the coumarin derivatives K-679 and K-680 almost completely suppressed TNFα release in monocytes; two isoxazole compounds (ISO-1 and ISO-66) as well as the Schiff base compound K-664.1 exhibited moderate suppression of TNFα release; whereas the chromene-derived T-614, isoxazole ISO-92, carbonyl oxime OXIM-11, and hormone isomer d-T4 almost completely spared TNFα release at concentrations up to 50 μM (FIG. 1A). The release of the chemokines MCP-1 and IL-8 segregated similarly, with some exceptions: ISO-92 and K664.1 were both stronger suppressors of MCP-1 than TNFα; d-T4 exhibited moderate suppression of IL-8 compared to sparing of TNFα; and ISO-66 spared IL-8 despite moderately suppressing TNFα (FIG. 1B).

Since this bioassay only tests broad inflammatory activity in response to LPS administration, it was sought to determine the role of MIF. Surprisingly, it was found that peripheral blood cells isolated from wild-type and MIF−/− animals exhibited no significant differences in their release of TNFα and IL-6 in response to LPS, suggesting that this assay may test a totally MIF-independent bioactivity (FIG. 1C). This was confirmed by selecting one of the highly anti-inflammatory molecules identified in the screen, the coumarin derivative K-680. When applied to murine peripheral blood cells stimulated with LPS, K-680 demonstrated the same dose-dependent suppression of TNFα in wild-type and MIF−/− cells (FIG. 1D). This observation suggested that MIF inhibitors with a strong anti-inflammatory profile may have significant off-target effects, while cytokine sparing compounds may be more MIF specific. Both T-614 and OXIM-11 exhibited minimal suppression of the cytokines analyzed in this study; of these, it was elected to investigate T-614 further, since it is a drug in clinical use with greater potential for clinical applications based on the previously unknown qualities.

T-614 Inhibits MIF in vitro: Multiple approaches have been used to examine MIF activity in vitro, including measurement of glucocorticoid override and ERK phosphorylation, but these assays can produce inconsistent results and may involve non-MIF effects. To examine T-614 as a selective MIF inhibitor, two bioassays were chosen where the readout is directly elicited by exogenously added rMIF protein. In the first assay, rMIF was found to significantly increase BrdU incorporation in synchronized Raji B cells at a concentration of 1 ng/mL, which mirrors the findings of Leng et al. using thymidine incorporation in the same cells (5). T-614 did not affect BrdU incorporation on its own at concentrations as high as 200 uM, which similar to previous observations (39); however, it did attenuate the rMIF effect at a concentration of 100 uM (FIG. 2A). For the second bioassay, IL-8 production was analyzed in adherence-purified peripheral blood monocytes, where we observed 100 ng/mL rMIF to trigger a two-fold increase in IL-8 released over 24 hours. Although T-614 did not affect IL-8 on its own and did not attenuate IL-8 release from LPS stimulation in enriched monocytes (FIG. 1B), it did dose-dependently suppress exogenous rMIF-induced IL-8 release from adherence-purified monocytes (FIG. 2B).

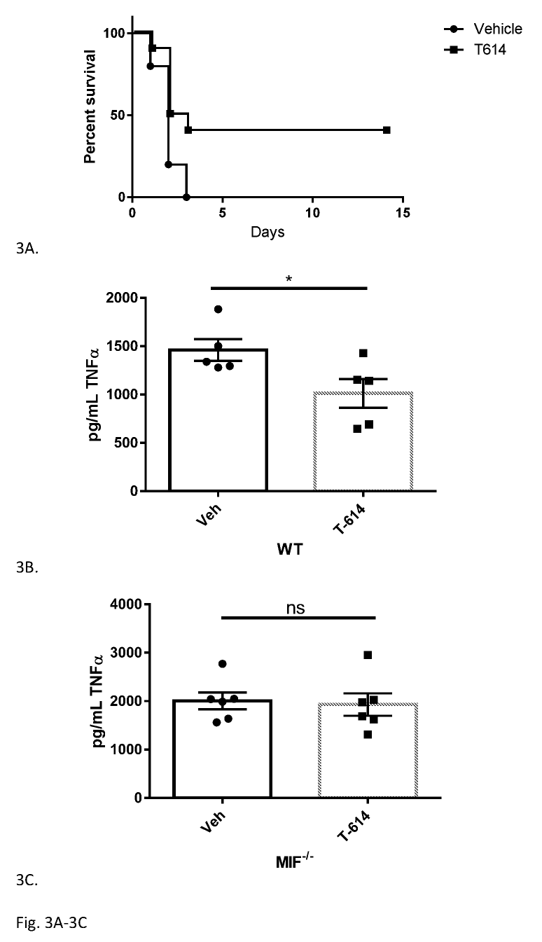

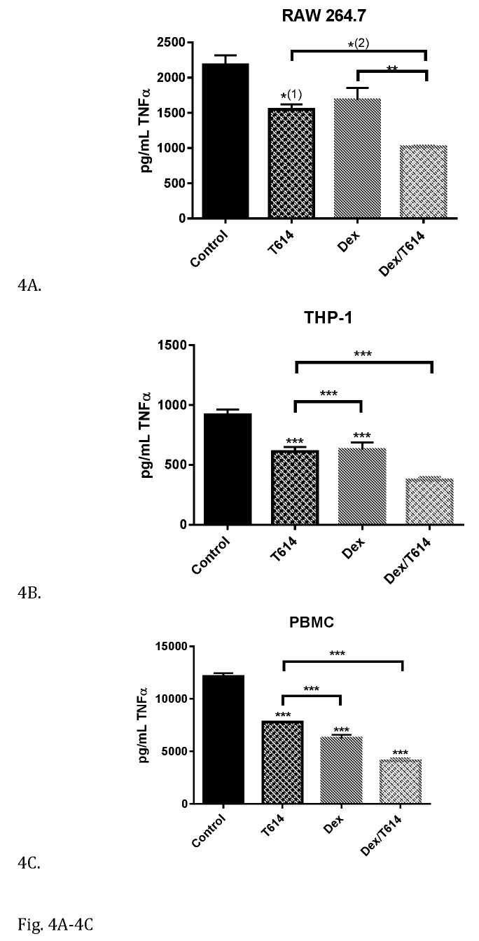

T-614 Inhibits MIF in vivo—In order to test in vivo efficacy and selectivity of T-614 as an MIF inhibitor, a murine endotoxemia model was employed that has been well characterized in the context of MIF using both knockout and inhibitory approaches (8, 22, 26). Using BALB/c mice that are vulnerable to endotoxemia, T-614-treated mice showed significantly increased survival after a lethal dose of LPS compared to vehicle-treated controls (FIG. 3A). T-614 treatment also attenuated TNFα release measured in serum isolated ninety minutes post-LPS administration in wild-type C57/BL6 mice; in MIF^{-/-} mice, however, there was no significant effect of T-614 on serum TNFα ninety minutes post-LPS administration (FIG. 3B). These data suggest that T-614 can attenuate a systemic inflammatory response in vivo, and that this effect is mediated by MIF.

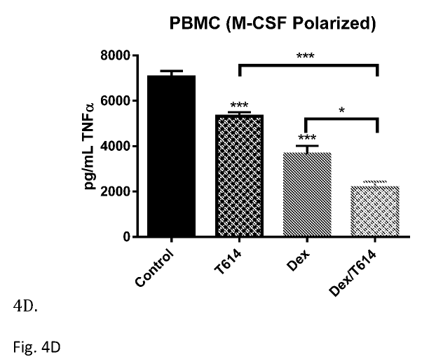

T-614 Synergizes with Glucocorticoids—Glucocorticoid synergy has been demonstrated in the context of MIF in previous studies using RNA silencing and anti-MIF antibodies; however, no studies have examined anti-MIF small molecules in this context (17, 21). Since these studies used murine RAW 264.7 macrophage and human THP-1 monocyte cell lines, similar systems were adapted for this study. For RAW 264.7 cells, individual pretreatment with inhibitor and dexamethasone significantly attenuated TNFα release induced by four-hour stimulation with LPS, and the combination of the two drugs had an additive effect (FIG. 4A). A similar additive effect was observed in the setting of THP-1 cells stimulated with LPS for 16 hours (FIG. 4B). To confirm the effect in primary cells, adherence-purified human peripheral blood monocytes were also tested as well as M-CSF polarized macrophages and again it was found that both T-614 and dexamethasone can individually attenuate LPS-induced TNFα release, and that the combination suppressed it further (FIG. 4C). It was noted that although T-614 was generally cytokine-sparing up to 50 uM in monocytes enriched by magnetic selection (FIG. 1A), in the context of adherence-purified monocytes the drug did suppress TNFα at concentrations as low as 10 uM (FIG. 4C). These variations might be attributable to differences between these monocyte preparations, and, indeed, it has been reported that the standard adherence protocol yields a relatively lower purity monocyte population that may have distinct inflammatory responses (40, 41). It was also noted that T-614's cytokine sparing effects are preserved in M-CSF polarized macrophages up to 50 μM (FIG. 4C).

The desire for specificity in drugs has evolved over the last few decades. Although a “one target, one drug” approach was appealing for many years, it has become clear that there is a limited number of ligand-binding pockets among proteins, and almost all endogenous ligands (and therefore, drugs) must interact with multiple targets (42, 43). Nevertheless, there is still benefit to isolating drugs that lack particular off-target effects. Suppression of TNFα, for example, may be desirable in a general anti-inflammatory drug; however, several studies have shown that even this classical pro-inflammatory cytokine can have protective roles in tissue repair and regeneration. In the central nervous system, TNFα has been shown to induce proliferation of neural stem cells, likely via interactions with TNFR2 (44-46). Treatment with anti-TNFα has been linked to the development of demyelinating disease, which may relate to TNFα's roles in repair and neurogenesis (47-49). It stands to reason that when developing a drug for use in multiple sclerosis, such as an MIF inhibitor (50), it would be useful to design the drug to be TNFα-sparing in order to avoid worsening demyelinating disease.

The study here determined that diverse compounds with MIF-inhibitory activity segregate into at least three populations when tested in a broad in vitro inflammatory assay, LPS-stimulation of monocytes (FIG. 1A). It was further determined that anti-inflammatory activity in this assay was likely MIF-independent, since the most anti-inflammatory compounds—coumarin analogs K-679 and K-680—exhibited anti-inflammatory activity even in the absence of MIF (FIG. 1D). In view of these data, cytokine-sparing compounds can be selective MIF inhibitors, including the chromene derivative T-614.

Compound T-614, better known as iguratimod, was created in the late 1980s and characterized as an anti-inflammatory drug in the early 1990s. Early communications indicated that the drug is orally bioavailable and capable of attenuating edema and joint destruction in arthritis models as well as exhibiting analgesic properties (51). These effects were viewed as potentially related to cyclooxygenase inhibition, since T-614 inhibits both the activity and transcription of COX-2; however, T-614's mechanism of action seems distinct from standard non-steroidal anti-inflammatory drugs (NSAIDs) (52, 53). Notably, one study found that T-614 inhibits both release and intracellular accumulation of the cytokine IL-1β in LPS-stimulated human peripheral blood monocytes, whereas the NSAID indomethacin inhibits release but increases intracellular accumulation of this cytokine (54). T-614 was also found to inhibit production of cytokines such as TNFα, IL-1β, IL-6, IL-8, and MCP-1 in a variety of cell types (54-57). Over time several more activities were attributed to T-614, including inhibition of immunoglobulin production by B cells (58), suppression of the IL-17 axis (59, 60), and promotion of bone anabolism by modulation of both osteoblastic and osteoclastic differentiation (61, 62). Despite these observations, no study has identified a molecular target for this drug, a daunting task given the diverse activities involved (63).

In this study, a molecular target has been identified for the first time that may explain some of the observed activities for T-614. T-614 interacts with the MIF trimer, inhibiting MIF's tautomerase enzymatic activity with an IC₅₀ comparable to ISO-1, the most commonly used MIF inhibitor (30) (Table 1). This interaction is relevant to MIF biology, since T-614 was able to inhibit MIF-induced proinflammatory effects including proliferation of B cells and cytokine release from monocytes (FIG. 2A, 2B). Moreover, these effects are selective for MIF, since T-614 was relatively cytokine-sparing in the specificity-guided screen (FIG. 1A) and did not suppress systemic inflammation in the absence of MIF (FIG. 3B). All these data suggest that T-614's anti-inflammatory effects may be mediated partially or entirely through MIF inhibition.

MIF is a pleiotropic molecule, and MIF inhibition could potentially underlie other observed activities of T-614. For example, MIF may be involved in T-614-mediated inhibition of immunoglobulin production from B cells: MIF has been previously linked to immunoglobulin production (64) and has a well-known role in promoting survival of B cells (65). T-614 is known to suppress the IL-17 signaling axis; MIF is known to stimulate this axis (66). T-614 has been shown to inhibit osteoclastic differentiation, which is induced by MIF (67, 68). It is not difficult to imagine that MIF may be the target of T-614 in all of these activities, and responsible for some or all of the efficacy of T-614 as a disease-modifying anti-rheumatic drug. Of further note, several studies have found that MIF has a significant role in the pathogenesis of rheumatoid arthritis and may be a relevant target in the disease (9, 69-72).

In addition to establishing T-614 as an MIF-targeting molecule, also provided is evidence for novel clinical applications of this drug. Also known as iguratimod, T-614 is currently clinically available in Japan and China as a daily oral formulation administered at 25-50 mg daily, which has shown safety and efficacy in improving symptoms and disease progression in rheumatoid arthritis both as a monotherapy (73-75) and in combination with methotrexate (76). Several preclinical studies have suggested that the drug may also have utility in other settings, such as multiple sclerosis (77) and cachexia in the context of adenocarcinoma (78). MIF is an influential player in a large variety of disease processes, including autoimmune (10, 11, 79, 80), neurologic (81, 82), metabolic (83), and oncologic conditions (84-87). Several of these disease processes benefit from treatment with glucocorticoids, of which MIF is an endogenous counter-regulator (16). Here it is shown that T-614, acting as an MIF inhibitor, can synergize with glucocorticoids in an in vitro inflammatory model (FIG. 4A-C). Since it has also been determined herein that T-614 is cytokine-sparing (and especially TNFα-sparing) compared to other MIF inhibitors, it would likely be a treatment for multiple sclerosis, where TNFα inhibition may exacerbate demyelinating disease (47).

In sum, the findings highlight the importance of considering off-target effects in drug development, since it was observed that even highly affine MIF inhibitors have distinct anti-inflammatory effects in vitro. Use of a specificity-guided screen highlighted the clinically available chromene derivative compound T-614 (iguratimod) as a selective MIF inhibitor, which further investigation revealed could have use as a steroid-sparing therapeutic.

### Example 2

Congenital diaphragmatic hernia (CDH) is a complex birth anomaly, associated with lung hypoplasia and persistent pulmonary hypertension of the newborn (PPHN). Despite advances in neonatal care and new modalities of treatment, CDH is associated with average 50% mortality. So far, there is no antenatal therapeutic approach to limit CDH mortality and morbidity. Herein is disclosed a therapy for CDH based on iguratimod administration.

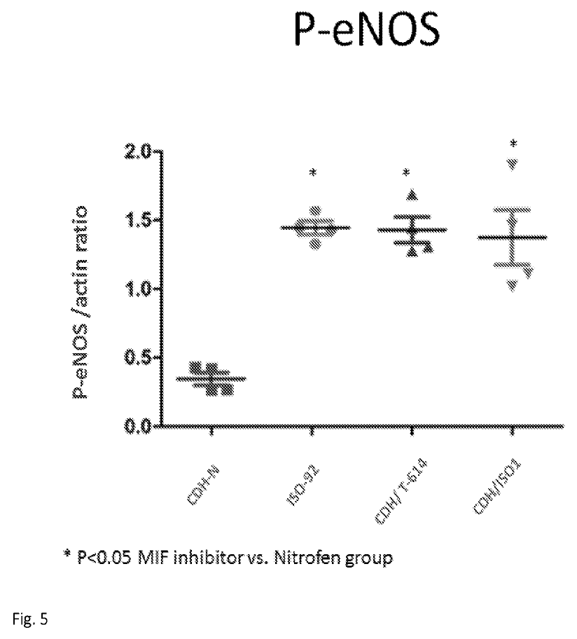

As shown in FIG. 5, a significant increase of eNOS phosphorylation was induced by MIF inhibitors (all, that were tested, namely, ISO-1, ISO-92 and the proposed MIF inhibitor T614/iguratimod) in comparison to Nitrofen group among neonates pups with CDH (P<0.05). This biological significant increase of P-eNOS has a main role in alleviating the severity of pulmonary hypertension after birth among these pups with CDH (Vasodilator effect induced by increase NO synthesis).

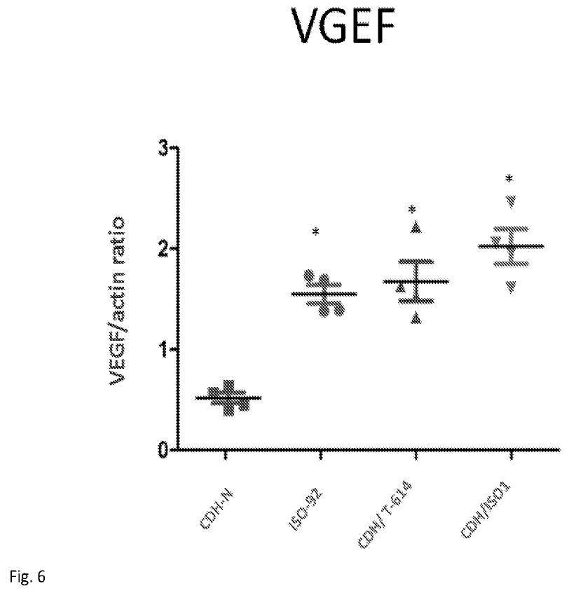

A significant increase of VGEF was seen among neonates pups with CDH and treated by MIF inhibitors (ISO-1, ISO-92 and the proposed MIF inhibitor T614/iguratimod) in comparison to Nitrofen group with CDH (P<0.05), as shown in FIG. 6. An increase in VGEF expression is the main cause of inducement of angiogenesis followed by induction of lung development among treated groups with MIF inhibitor compared to non-treated group (nitrofen group), as shown by CT lung volumes studies.

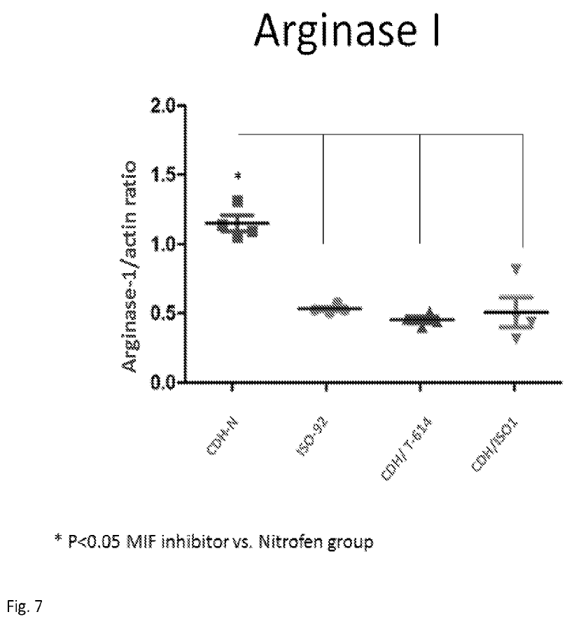

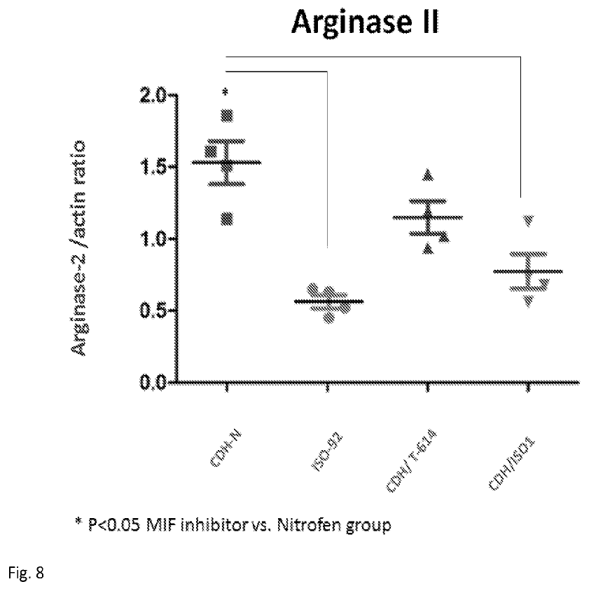

FIGS. 7 and 8 show Arginase I and II studies, respectively. A significant reduction of Arginase I was seen among treated pups with CDH with MIF inhibitors (including T614) compared to Nitrofen group (P<0.05) (FIG. 7), while Arginase II was reduced in all treated groups but was significantly lower among ISO-92 and ISO-1 only (P<0.05) (FIG. 8). Increases in both Arginase I& II lead to marked reduction of NO synthesis by diverting L-arginine (NO precursor), used in a different metabolic pathway (L-orthinine synthesis), which leads to marked reduction of NO bioavailability.

The results clearly indicate a therapeutic use for T614/iguratimod in treating CDH.

### Example 3

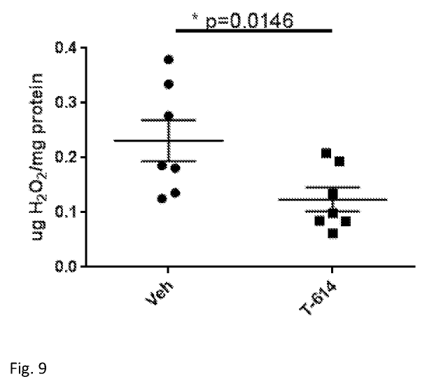

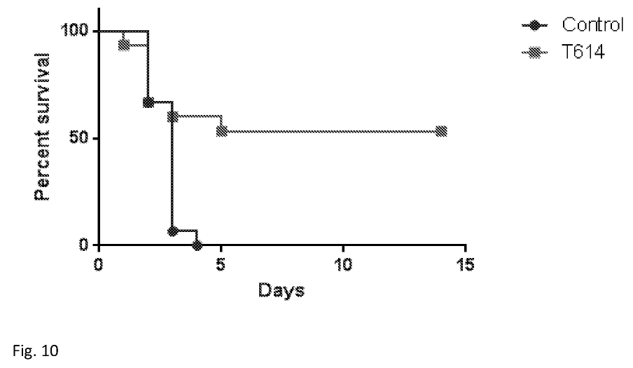

T614 can be used to reduce hepatoxicity resulting from acetaminophen overdose. This is demonstrated using an acetaminophen (APAP)-induced hepatotoxicity model. See FIGS. 9 and 10. For all experiments, animals were fasted for 16 hours by transfer to clean cages without food prior to dosing with APAP. APAP was administered by intraperitoneal injection of a 15 mg/mL solution in warm 0.9% saline (Hospira, Lake Forest, Ill.). Injection volumes were adjusted to mouse weight and volumes up to 0.8 mL were well tolerated. After APAP administration, food was provided ad libitum. For survival experiments (see FIG. 10), animals were dosed with 420 mg/kg APAP and monitored for two weeks; when applicable, T-614 (20 mg/kg) was administered intraperitoneally 1 hour pre-APAP, 6 hours post-APAP, and once daily in the morning for four days afterward. For acute toxicity experiments (see FIG. 9), animals were given a non-lethal dose of APAP (300 mg/kg) and euthanized at four hours post-APAP by CO₂ asphyxiation with cervical dislocation and blood and liver were harvested for analysis; when applicable, T-614 (20 mg/kg) was administered intraperitoneally 1 hour pre-APAP and 1 hour post-APAP. Blood was collected by cardiac puncture and allowed to clot for 20 minutes at room temperature and 20 minutes at 4° C.; sera were isolated by centrifugation at 300×g for 10 minutes and stored at −20° C. for further analysis. Livers were mobilized and divided into major lobes; left lobes were fixed in phosphate-buffered formalin (Fisher Scientific, Pittsburgh, Pa.), median lobes were processed for H₂O₂ studies or flash frozen in liquid N₂, and remaining lobes were flash frozen in liquid N₂. Liver H₂O₂ content was determined using a Hydrogen Peroxide Assay Kit from Abcam (Cambridge, United Kingdom); for this application, livers were homogenized by 15 passes in a Dounce homogenizer and deproteinized with perchloric acid as per the manufacturer's recommendation, and assays were performed on the same day as tissue isolation.

### REFERENCES

1. David, J. R. (1966) Delayed hypersensitivity in vitro: its mediation by cell-free substances formed by lymphoid cell-antigen interaction. Proceedings of the National Academy of Sciences of the United States of America. 56, 72-77
2. Bloom, B. R., and Bennett, B. (1966) Mechanism of a reaction in vitro associated with delayed-type hypersensitivity. Science. 153, 80-82
3. Calandra, T., and Roger, T. (2003) Macrophage migration inhibitory factor: a regulator of innate immunity. Nat Rev Immunol. 3, 791-800
4. Bernhagen, J., Krohn, R., Lue, H., Gregory, J. L., Zemecke, A., Koenen, R. R., Dewor, M., Georgiev, I., Schober, A., Leng, L., Kooistra, T., Fingerle-Rowson, G., Ghezzi, P., Kleemann, R., McColl, S. R., Bucala, R., Hickey, M. J., and Weber, C. (2007) MIF is a noncognate ligand of CXC chemokine receptors in inflammatory and atherogenic cell recruitment. Nat. Med. 13, 587-596
5. Leng, L., Metz, C. N., Fang, Y., Xu, J., Donnelly, S., Baugh, J., Delohery, T., Chen, Y., Mitchell, R. A., and Bucala, R. (2003) MIF Signal Transduction Initiated by Binding to CD74. Journal of Experimental Medicine. 197, 1467-1476
6. Mitchell, R. A., Metz, C. N., Peng, T., and Bucala, R. (1999) Sustained mitogen-activated protein kinase (MAPK) and cytoplasmic phospholipase A2 activation by macrophage migration inhibitory factor (MIF). Regulatory role in cell proliferation and glucocorticoid action. J. Biol. Chem. 274, 18100-18106
7. Shi, X., Leng, L., Wang, T., Wang, W., Du, X., Li, J., McDonald, C., Chen, Z., Murphy, J. W., Lolis, E., Noble, P., Knudson, W., and Bucala, R. (2006) CD44 is the signaling component of the macrophage migration inhibitory factor-CD74 receptor complex. Immunity. 25, 595-606
8. Bozza, M., Satoskar, A. R., Lin, G., Lu, B., Humbles, A. A., Gerard, C., and David, J. R. (1999) Targeted disruption of migration inhibitory factor gene reveals its critical role in sepsis. J. Exp. Med. 189, 341-346
9. Onodera, S., Kaneda, K., Mizue, Y., Koyama, Y., Fujinaga, M., and Nishihira, J. (2000) Macrophage migration inhibitory factor up-regulates expression of matrix metalloproteinases in synovial fibroblasts of rheumatoid arthritis. J. Biol. Chem. 275, 444-450
10. de Jong, Y. P., Abadia-Molina, A. C., Satoskar, A. R., Clarke, K., Rietdijk, S. T., Faubion, W. A., Mizoguchi, E., Metz, C. N., Alsahli, M., Hove, ten, T., Keates, A. C., Lubetsky, J. B., Farrell, R. J., Michetti, P., van Deventer, S. J., Lolis, E., David, J. R., Bhan, A. K., Terhorst, C., and Sahli, M. A. (2001) Development of chronic colitis is dependent on the cytokine MIF. Nature Immunology. 2, 1061-1066
11. Powell, N. D., Papenfuss, T. L., and McClain, M. A. (2005) Cutting edge: macrophage migration inhibitory factor is necessary for progression of experimental autoimmune encephalomyelitis. The Journal of . . . . 10.4049/jimmuno1.175.9.5611
12. Curtis, J. R., Westfall, A. O., Allison, J., Bijlsma, J. W., Freeman, A., George, V., Kovac, S. H., Spettell, C. M., and Saag, K. G. (2006) Population-based assessment of adverse events associated with long-term glucocorticoid use. Arthritis Rheum. 55, 420-426
13. Huscher, D., Thiele, K., Gromnica-Ihle, E., Hein, G., Demary, W., Dreher, R., Zink, A., and Buttgereit, F. (2009) Dose-related patterns of glucocorticoid-induced side effects. Annals of the Rheumatic Diseases. 68, 1119-1124
14. Jiang, C.-L., Liu, L., Li, Z., and Buttgereit, F. (2015) The novel strategy of glucocorticoid drug development via targeting nongenomic mechanisms. STEROIDS. 102, 27-31
15. Schacke, H., Schottelius, A., Docke, W.-D., Strehlke, P., Jaroch, S., Schmees, N., Rehwinkel, H., Hennekes, H., and Asadullah, K. (2004) Dissociation of transactivation from transrepression by a selective glucocorticoid receptor agonist leads to separation of therapeutic effects from side effects. Proceedings of the National Academy of Sciences of the United States of America. 101, 227-232
16. Calandra, T., Bernhagen, J., Metz, C. N., Spiegel, L. A., Bacher, M., Donnelly, T., Cerami, A., and Bucala, R. (1995) MIF as a glucocorticoid-induced modulator of cytokine production. Nature. 377, 68-71
17. Roger, T., Chanson, A.-L., Knaup-Reymond, M., and Calandra, T. (2005) Macrophage migration inhibitory factor promotes innate immune responses by suppressing glucocorticoid-induced expression of mitogen-activated protein kinase phosphatase-1. Eur. J. Immunol. 35, 3405-3413
18. Fan, H., Kao, W., Yang, Y. H., Gu, R., Harris, J., Fingerle-Rowson, G., Bucala, R., Ngo, D., Beaulieu, E., and Morand, E. F. (2014) Macrophage Migration Inhibitory Factor inhibits the anti-inflammatory effects of glucocorticoids via glucocorticoid-induced leucine zipper. Arthritis & Rheumatology. 10.1002/art.38689
19. Petrovsky, N., Socha, L., Silva, D., Grossman, A. B., Metz, C., and Bucala, R. (2003) Macrophage migration inhibitory factor exhibits a pronounced circadian rhythm relevant to its role as a glucocorticoid counter-regulator. Immunol. Cell Biol. 81, 137-143
20. Flaster, H., Bernhagen, J., Calandra, T., and Bucala, R. (2007) The Macrophage Migration Inhibitory Factor-Glucocorticoid Dyad: Regulation of Inflammation and Immunity. Molecular Endocrinology. 21, 1267-1280
21. Kerschbaumer, R. J., Rieger, M., Völkel, D., Le Roy, D., Roger, T., Garbaraviciene, J., Boehncke, W.-H., Müllberg, J., Hoet, R. M., Wood, C. R., Antoine, G., Thiele, M., Savidis-Dacho, H., Dockal, M., Ehrlich, H., Calandra, T., and Scheiflinger, F. (2012) Neutralization of macrophage migration inhibitory factor (MIF) by fully human antibodies correlates with their specificity for the (3-sheet structure of MIF. Journal of Biological Chemistry. 287, 7446-7455
22. Bacher, M., Meinhardt, A., Lan, H. Y., Mu, W., Metz, C. N., Chesney, J. A., Calandra, T., Gemsa, D., Donnelly, T., Atkins, R. C., and Bucala, R. (1997) Migration inhibitory factor expression in experimentally induced endotoxemia. Am. Pathol. 150, 235-246
23. Harper, J. M., Wilkinson, J. E., and Miller, R. A. (2010) Macrophage migration inhibitory factor-knockout mice are long lived and respond to caloric restriction. FASEB J. 24, 2436-2442
24. Rosengren, E., Bucala, R., Aman, P., Jacobsson, L., Odh, G., Metz, C. N., and Rorsman, H. (1996) The immunoregulatory mediator macrophage migration inhibitory factor (MIF) catalyzes a tautomerization reaction. Mol. Med. 2, 143-149
25. Lubetsky, J. B. (2002) The Tautomerase Active Site of Macrophage Migration Inhibitory Factor Is a Potential Target for Discovery of Novel Anti-inflammatory Agents. Journal of Biological Chemistry. 277, 24976-24982
26. Al-Abed, Y. (2005) ISO-1 Binding to the Tautomerase Active Site of MIF Inhibits Its Pro-inflammatory Activity and Increases Survival in Severe Sepsis. Journal of Biological Chemistry. 280, 36541-36544
27. Healy, Z. R., Liu, H., Holtzclaw, W. D., and Talalay, P. (2011) Inactivation of tautomerase activity of macrophage migration inhibitory factor by sulforaphane: a potential biomarker for anti-inflammatory intervention. Cancer Epidemiol. Biomarkers Prev. 20, 1516-1523
28. Cheng, K.-F., and Al-Abed, Y. (2006) Critical modifications of the ISO-1 scaffold improve its potent inhibition of macrophage migration inhibitory factor (MIF) tautomerase activity. Bioorganic & Medicinal Chemistry Letters. 16, 3376-3379
29. Crichlow, G. V., Cheng, K. F., Dabideen, D., Ochani, M., Aljabari, B., Pavlov, V. A., Miller, E. J., Lolis, E., and Al-Abed, Y. (2007) Alternative Chemical Modifications Reverse the Binding Orientation of a Pharmacophore Scaffold in the Active Site of Macrophage Migration Inhibitory Factor. Journal of Biological Chemistry. 282, 23089-23095
30. Al-Abed, Y., and VanPatten, S. (2011) MIF as a disease target: ISO-1 as a proof-of-concept therapeutic. Future Medicinal Chemistry. 3, 45-63
31. Orita, M., Yamamoto, S., Katayama, N., Aoki, M., Takayama, K., Yamagiwa, Y., Seki, N., Suzuki, H., Kurihara, H., Sakashita, H., Takeuchi, M., Fujita, S., Yamada, T., and Tanaka, A. (2001) Coumarin and Chromen-4-one Analogues as Tautomerase Inhibitors of Macrophage Migration Inhibitory Factor: Discovery and X-ray Crystallography. J. Med. Chem. 44, 540-547
32. Orita, M., Yamamoto, S., Katayama, N., and Fujita, S. (2002) Macrophage migration inhibitory factor and the discovery of tautomerase inhibitors. Curr. Pharm. Des. 8, 1297-1317
33. Dios, A., Mitchell, R. A., Aljabari, B., Lubetsky, J., O'Connor, K., Liao, H., Senter, P. D., Manogue, K. R., Lolis, E., Metz, C., Bucala, R., Callaway, D. J. E., and Al-Abed, Y. (2002) Inhibition of MIF Bioactivity by Rational Design of Pharmacological Inhibitors of MIF Tautomerase Activity. J. Med. Chem. 45, 2410-2416
34. Al-Abed, Y., Metz, C. N., Cheng, K. F., Aljabari, B., VanPatten, S., Blau, S., Lee, H., Ochani, M., Pavlov, V. A., Coleman, T., Meurice, N., Tracey, K. J., and Miller, E. J. (2011) Thyroxine is a potential endogenous antagonist of macrophage migration inhibitory factor (MIF) activity. PNAS. 108, 8224-8227
35. Kudrin, A., Scott, M., Martin, S., Chung, C.-W., Donn, R., McMaster, A., Ellison, S., Ray, D., Ray, K., and Binks, M. (2006) Human macrophage migration inhibitory factor: a proven immunomodulatory cytokine? J. Biol. Chem. 281, 29641-29651
36. Hudson, J. D., Shoaibi, M. A., Maestro, R., Carnero, A., Hannon, G. J., and Beach, D. H. (1999) A proinflammatory cytokine inhibits p53 tumor suppressor activity. J. Exp. Med. 190, 1375-1382
37. Dickerhof, N., Schindler, L., Bernhagen, J., Kettle, A. J., and Hampton, M. B. (2015) Macrophage migration inhibitory factor (MIF) Is Rendered enzymatically inactive by myeloperoxidase-derived Oxidants but Retains Its immunomodulatory function. Free Radical Biology and Medicine. 10.1016/j.freeradbiomed.2015.09.009
38. Repetto, G., del Peso, A., and Zurita, J. L. (2008) Neutral red uptake assay for the estimation of cell viability/cytotoxicity. Nat Protoc. 3, 1125-1131
39. Yamamoto, T., Aikawa, Y., Funaki, J., and Tanaka, K. (2007) Immunopharmacological studies of a disease-modifying antirheumatic drug Iguratimod (T-614)—Its effect on immunoglobulin production and lymphocyte proliferation. JAPANESE . . . .
40. Zhou, L., Somasundaram, R., Nederhof, R. F., Dijkstra, G., Faber, K. N., Peppelenbosch, M. P., and Fuhler, G. M. (2012) Impact of Human Granulocyte and Monocyte Isolation Procedures on Functional Studies. Clinical and Vaccine Immunology. 19, 1065-1074
41. Johnston, L., Harding, S. A., and La Flamme, A. C. (2015) Comparing methods for ex vivo characterization of human monocyte phenotypes and in vitro responses. Immunobiology. 220, 1305-1310
42. Medina-Franco, J. L., Giulianotti, M. A., Welmaker, G. S., and Houghten, R. A. (2013) Shifting from the single to the multitarget paradigm in drug discovery. Drug Discovery Today. 18, 495-501
43. Skolnick, J., Gao, M., Roy, A., Srinivasan, B., and Zhou, H. (2015) Implications of the small number of distinct ligand binding pockets in proteins for drug discovery, evolution and biochemical function. Bioorganic & Medicinal Chemistry Letters. 25, 1163-1170
44. Wu, J. P., Kuo, J. S., Liu, Y. L., and Tzeng, S. F. (2000) Tumor necrosis factor-alpha modulates the proliferation of neural progenitors in the subventricular/ventricular zone of adult rat brain. Neurosci. Lett. 292, 203-206
45. Widera, D., Mikenberg, I., Elvers, M., Kaltschmidt, C., and Kaltschmidt, B. (2006) Tumor necrosis factor alpha triggers proliferation of adult neural stem cells via IKK/NF-kappaB signaling. BMC Neurosci. 7, 64
46. Iosif, R. E. (2006) Tumor Necrosis Factor Receptor 1 Is a Negative Regulator of Progenitor Proliferation in Adult Hippocampal Neurogenesis. J. Neurosci. 26, 9703-9712
47. Matsumoto, T., Nakamura, I., Miura, A., Momoyama, G., and Ito, K. (2012) New-onset multiple sclerosis associated with adalimumab treatment in rheumatoid arthritis: a case report and literature review. Clin Rheumatol. 32, 271-275
48. Kaltsonoudis, E., Voulgari, P. V., Konitsiotis, S., and Drosos, A. A. (2014) Demyelination and other neurological adverse events after anti-TNF therapy. Autoimmunity Reviews. 13, 54-58
49. Dooley, D., Vidal, P., and Hendrix, S. (2014) Immunopharmacological intervention for successful neural stem cell therapy: New perspectives in CNS neurogenesis and repair. Pharmacology and Therapeutics. 141, 21-31
50. Ji, N., Kovalovsky, A., Fingerle-Rowson, G., Guentzel, M. N., and Forsthuber, T. G. (2015) Macrophage migration inhibitory factor promotes resistance to glucocorticoid treatment in EAE. Neurology: Neuroimmunology & Neuroinflammation. 2, e139-e139
51. Tanaka, K., Shimotori, T., Makino, S., and Aikawa, Y. (1992) Pharmacological studies of the new antiinflammatory agent 3-formylamino-7-methylsulfonylamino-6-phenoxy-4H-1-benzopyran-4-one. 1st communication: . . . Arzneimittel- . . . .
52. Tanaka, K., Makino, S., Shimotori, T., and Aikawa, Y. (1992) Pharmacological studies of the new antiinflammatory agent 3-formylamino-7-methylsulfonylamino-6-phenoxy-4′-1-benzopyran-4-one. 2nd communication: effect on . . . Arzneimittel- . . . .
53. Tanaka, K., Kawasaki, H., Kurata, K., Aikawa, Y., Tsukamoto, Y., and Inaba, T. (1995) T-614, a novel antirheumatic drug, inhibits both the activity and induction of cyclooxygenase-2 (COX-2) in cultured fibroblasts. Jpn. J. Pharmacol. 67, 305-314
54. Tanaka, K., Aikawa, Y., Kawasaki, H., Asaoka, K., Inaba, T., and Yoshida, C. (1992) Pharmacological studies on 3-formylamino-7-methylsulfonylamino-6-phenoxy-4H-1-benzopyran-4-one (T-614), a novel antiinflammatory agent. 4th communication: inhibitory effect on the production of interleukin-1 and interleukin-6. J. Pharmacobio-dyn. 15, 649-655
55. Aikawa, Y., Yamamoto, M., Yamamoto, T., Morimoto, K., and Tanaka, K. (2002) An anti-rheumatic agent T-614 inhibits NF-kappaB activation in LPS- and TNF-alpha-stimulated THP-1 cells without interfering with IkappaBalpha degradation. Inflamm. res. 51, 188-194
56. Kohno, M., Aikawa, Y., Tsubouchi, Y., Hashiramoto, A., Yamada, R., Kawahito, Y., Inoue, K., Kusaka, Y., Kondo, M., and Sano, H. (2001) Inhibitory effect of T-614 on tumor necrosis factor-alpha induced cytokine production and nuclear factor-kappaB activation in cultured human synovial cells. J Rheumatol. 28, 2591-2596
57. Kawakami, A., Tsuboi, M., Urayama, S., Matsuoka, N., Yamasaki, S., Hida, A., Aoyagi, T., Furuichi, I., Nakashima, T., Migita, K., Kawabe, Y., Nakashima, M., Origuchi, T., and Eguchi, K. (1999) Inhibitory effect of a new anti-rheumatic drug T-614 on costimulatory molecule expression, cytokine production, and antigen presentation by synovial cells. J. Lab. Clin. Med. 133, 566-574
58. Tanaka, K., Yamamoto, T., Aikawa, Y., Kizawa, K., Muramoto, K., Matsuno, H., and Muraguchi, A. (2003) Inhibitory effects of an anti-rheumatic agent T-614 on immunoglobulin production by cultured B cells and rheumatoid synovial tissues engrafted into SCID mice. Rheumatology (Oxford). 42, 1365-1371
59. Luo, Q., Sun, Y., Liu, W., Qian, C., Jin, B., Tao, F., Gu, Y., Wu, X., Shen, Y., and Xu, Q. (2013) A novel disease-modifying antirheumatic drug, iguratimod, ameliorates murine arthritis by blocking IL-17 signaling, distinct from methotrexate and leflunomide. The Journal of Immunology. 191, 4969-4978
60. Wei, Y., Sun, X., Hua, M., Tan, W., Wang, F., and Zhang, M. (2015) Research Articlelnhibitory Effect of a Novel Antirheumatic Drug T-614 on the IL-6-Induced RANKL/OPG, IL-17, and MMP-3 Expression in Synovial Fibroblasts from Rheumatoid Arthritis Patients. BioMed Research International. 10.1155/2015/214683
61. Kuriyama, K., Higuchi, C., Tanaka, K., Yoshikawa, H., and Itoh, K. (2002) A novel anti-rheumatic drug, T-614, stimulates osteoblastic differentiation in vitro and bone morphogenetic protein-2-induced bone formation in vivo. Biochemical and Biophysical Research Communications. 299, 903-909
62. Gan, K., Yang, L., Xu, L., Feng, X., Zhang, Q., Wang, F., Tan, W., and Zhang, M. (2016) Iguratimod (T-614) suppresses RANKL-induced osteoclast differentiation and migration in RAW264.7 cells via NF-κB and MAPK pathways. International Immunopharmacology. 35, 294-300
63. Tanaka, K. (2009) Iguratimod (T-614): A novel disease modifying anti-rheumatic drug. Rheumatol Rep. 1, e4
64. Bacher, M., Metz, C. N., Calandra, T., Mayer, K., Chesney, J., Lohoff, M., Gemsa, D., Donnelly, T., and Bucala, R. (1996) An essential regulatory role for macrophage migration inhibitory factor in T-cell activation. Proceedings of the National Academy of Sciences of the United States of America. 93, 7849-7854
65. Gore, Y., Starlets, D., Maharshak, N., Becker-Herman, S., Kaneyuki, U., Leng, L., Bucala, R., and Shachar, I. (2008) Macrophage migration inhibitory factor induces B cell survival by activation of a CD74-CD44 receptor complex. J. Biol. Chem. 283, 2784-2792
66. Stojanovic, I., Cvjetićanin, T., Lazaroski, S., Stosic-Grujicic, S., and Miljkovic, D. (2009) Macrophage migration inhibitory factor stimulates interleukin-17 expression and production in lymph node cells. Immunology. 126, 74-83
67. Madeira, M. F. M., Queiroz-Junior, C. M., Costa, G. M., Santos, P. C., Silveira, E. M., Garlet, G. P., Cisalpino, P. S., Teixeira, M. M., Silva, T. A., and da Gloria Souza, D. (2012) MIF induces osteoclast differentiation and contributes to progression of periodontal disease in mice. Microbes Infect. 14, 198-206
68. Gu, R., Santos, L. L., Ngo, D., Fan, H., Singh, P. P., Fingerle-Rowson, G., Bucala, R., Xu, J., Quinn, J. M. W., and Morand, E. F. (2015) Macrophage migration inhibitory factor is essential for osteoclastogenic mechanisms in vitro and in vivo mouse model of arthritis. Cytokine. 72, 135-145
69. Mikulowska, A., Metz, C. N., Bucala, R., and Holmdahl, R. (1997) Macrophage migration inhibitory factor is involved in the pathogenesis of collagen type II-induced arthritis in mice. J. Immunol. 158, 5514-5517
70. Santos, L., Hall, P., Metz, C., Bucala, R., and Morand, E. F. (2001) Role of macrophage migration inhibitory factor (MIF) in murine antigen-induced arthritis: interaction with glucocorticoids. Clin. Exp. Immunol. 123, 309-314
71. Onodera, S., Nishihira, J., Koyama, Y., Majima, T., Aoki, Y., Ichiyama, H., Ishibashi, T., and Minami, A. (2004) Macrophage migration inhibitory factor up-regulates the expression of interleukin-8 messenger RNA in synovial fibroblasts of rheumatoid arthritis patients: Common transcriptional regulatory mechanism between interleukin-8 and interleukin-1? Arthritis Rheum. 50, 1437-1447
72. Morand, E. F., Leech, M., and Bernhagen, J. (2006) MIF: a new cytokine link between rheumatoid arthritis and atherosclerosis. Nat Rev Drug Discov. 5, 399-411
73. Lü, L.-J., Teng, J.-L., Bao, C.-D., Han, X.-H., Sun, L.-Y., Xu, J.-H., Li, X.-F., and Wu, H.-X. (2008) Safety and efficacy of T-614 in the treatment of patients with active rheumatoid arthritis: a double blind, randomized, placebo-controlled and multicenter trial. Chin. Med. 1 121, 615-619
74. Lü, L.-J., Bao, C.-D., Dai, M., Teng, J.-L., Fan, W., Du, F., Yang, N.-P., Zhao, Y.-H., Chen, Z.-W., Xu, J.-H., He, P.-G., Wu, H.-X., Tao, Y., Zhang, M.-J., Han, X.-H., Li, X.-F., Gu, J.-R., Li, J.-H., and Yu, H. (2009) Multicenter, randomized, double-blind, controlled trial of treatment of active rheumatoid arthritis with T-614 compared with methotrexate. Arthritis Rheum. 61, 979-987
75. Okamura, K., Yonemoto, Y., Suto, T., Okura, C., and Takagishi, K. (2015) Efficacy at 52 weeks of daily clinical use of iguratimod in patients with rheumatoid arthritis. Mod Rheumatol. 25, 534-539
76. Duan, X.-W., Zhang, X.-L., Mao, S.-Y., Shang, J.-J., and Shi, X.-D. (2015) Efficacy and safety evaluation of a combination of iguratimod and methotrexate therapy for active rheumatoid arthritis patients: a randomized controlled trial. Clin Rheumatol. 10.1007/s10067-015-2999-6
77. Aikawa, Y., Tanuma, N., Shin, T., Makino, S., Tanaka, K., and Matsumoto, Y. (1998) A new anti-rheumatic drug, T-614, effectively suppresses the development of autoimmune encephalomyelitis. J. Neuroimmunol. 89, 35-42
78. Tanaka, K., Urata, N., Mikami, M., Ogasawara, M., Matsunaga, T., Terashima, N., and Suzuki, H. (2006) Effect of iguratimod and other anti-rheumatic drugs on adenocarcinoma colon 26-induced cachexia in mice. Inflamm. res. 56, 17-23
79. Lang, T., Foote, A., Lee, J. P. W., Morand, E. F., and Harris, J. (2015) MIF: Implications in the Pathoetiology of Systemic Lupus Erythematosus. Front. Immunol. 6, 115-10
80. Stosic-Grujicic, S., Stojanovic, I., Maksimovic-Ivanic, D., Momcilovic, M., Popadic, D., Harhaji, L., Miljkovic, D., Metz, C., Mangano, K., Papaccio, G., Al-Abed, Y., and Nicoletti, F. (2008) Macrophage migration inhibitory factor (MIF) is necessary for progression of autoimmune diabetes mellitus. J. Cell. Physiol. 215, 665-675
81. Bloom, J., and Al-Abed, Y. (2014) MIF: Mood Improving/Inhibiting Factor? J Neuroinflammation. 11, 11
82. Savaskan, N. E., Fingerle-Rowson, G., Buchfelder, M., and Eyüpoglu, I. Y. (2012) Brain Miffed by Macrophage Migration Inhibitory Factor. International Journal of Cell Biology. 2012, 1-11
83. Morrison, M. C., and Kleemann, R. (2015) Role of Macrophage Migration Inhibitory Factor in Obesity, Insulin Resistance, Type 2 Diabetes, and Associated Hepatic Co-Morbidities: A Comprehensive Review of Human and Rodent Studies. Front. Immunol. 6, 1-13
84. Tarnowski, M., Grymula, K., Liu, R., Tarnowska, J., Drukala, J., Ratajczak, J., Mitchell, R. A., Ratajczak, M. Z., and Kucia, M. (2010) Macrophage migration inhibitory factor is secreted by rhabdomyosarcoma cells, modulates tumor metastasis by binding to CXCR4 and CXCR7 receptors and inhibits recruitment of cancer-associated fibroblasts. Mol. Cancer Res. 8, 1328-1343
85. He, X. X., Chen, K., Yang, J., Li, X. Y., Gan, H. Y., and Liu, C. Y. (2009) Macrophage migration inhibitory factor promotes colorectal cancer. Molecular . . . . 10.2119/molmed.2008.00107
86. Chesney, J., Metz, C., Bacher, M., Peng, T., Meinhardt, A., and Bucala, R. (1999) An essential role for macrophage migration inhibitory factor (MIF) in angiogenesis and the growth of a murine lymphoma. Mol. Med. 5, 181-191
87. Chesney, J. A., and Mitchell, R. A. (2015) 25 Years On: A Retrospective on Migration Inhibitory Factor in Tumor Angiogenesis. Mol. Med. 21 Suppl 1, S19-24

1. A method of inhibiting macrophage migration inhibitory factor (MIF) in a subject comprising administering to the subject an amount of the following compound, or a pharmaceutically acceptable salt thereof, effective to inhibit MIF:

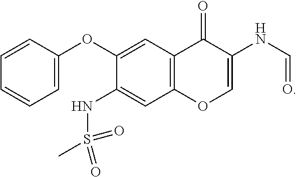 [SMILES: CS(=O)(=O)Nc1cc2occ(NC=O)c(=O)c2cc1Oc1ccccc1]

2. The method of claim 1, wherein the subject has congenital diaphragmatic hernia.

3. A method of inhibiting macrophage migration inhibitory factor (MIF) comprising contacting the MIF with an amount of the following compound, or a pharmaceutically acceptable salt thereof, effective to inhibit MIF:

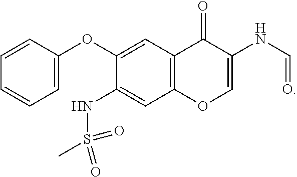 [SMILES: CS(=O)(=O)Nc1cc2occ(NC=O)c(=O)c2cc1Oc1ccccc1]

4-7. (canceled)

8. A method of reducing the dose of a steroid administered to a subject required to achieve a predetermined therapeutic effect in a disease treatable by steroid therapy, comprising administering to the subject having the disease an amount of a compound or a pharmaceutically acceptable salt of such compound effective to reduce the dose of steroid needed to achieve the therapeutic effect in the disease, wherein the compound has the following structure:

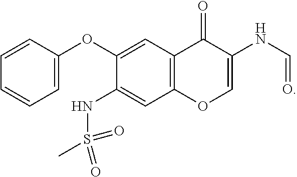 [SMILES: CS(=O)(=O)Nc1cc2occ(NC=O)c(=O)c2cc1Oc1ccccc1]

9-10. (canceled)

11. The method of claim 1, wherein the compound is administered.

12. The method of claim 1, wherein the pharmaceutically acceptable salt of the compound is administered.

13. The method of claim 1, wherein the subject is a human.

14. A composition comprising a pharmaceutically acceptable carrier, an amount of acetaminophen, and an amount of a compound having the structure set forth below, or a pharmaceutically acceptable salt of such compound:

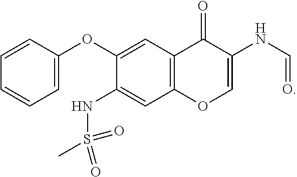 [SMILES: CS(=O)(=O)Nc1cc2occ(NC=O)c(=O)c2cc1Oc1ccccc1]

15-17. (canceled)

18. A method of decreasing the likelihood of hepatotoxicity in a subject resulting from an overdose of acetaminophen, comprising administering to the subject one or more doses of the acetaminophen composition of claim 1 amounting to more than 3 g of acetaminophen in 24 hours, wherein the amount of the compound or pharmaceutically acceptable salt of such compound is effective to decrease the likelihood of hepatotoxicity in a subject resulting from an overdose of acetaminophen.

19. (canceled)
